(19) United States
(12) Patent Application Publication
(10) Pub. No.: US 2008/0256669 A1
(43) Pub. Date: Oct. 16, 2008

(54) Plants with Multiple Transgenes on a Chromosome

(75) Inventors: Bradon Fabbri, Chesterfield, MO (US); Stefan Bledig, Chesterfield, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(73) Assignee: MONSANTO COMPANY, St. Louis, MO (US)

(21) Appl. No.: 12/104,020

(22) Filed: Apr. 16, 2008

Related U.S. Application Data
(60) Provisional application No. 60/912,032, filed on Apr. 16, 2007.

(51) Int. Cl. A01H 5/00

(52) U.S. Cl. 800/300.1; 800/320.1; 800/314; 800/312; 800/306; 800/300; 800/302; 800/301

(57) ABSTRACT

A transgenic seed or plant of a corn, cotton, rapeseed or soybean species having a recombinant chromosome with multiple transgenes for imparting traits to said seed or its plant. Useful traits include herbicide tolerance, insect resistance, nematode resistance, viral resistance, tolerance to water deficit, tolerance to nitrogen deficit, enhanced amino acid level in seed, enhanced starch level in seed, enhanced oil level in seed, modified oil composition, and increased yield. Useful recombinant chromosomes are produced with centromere DNA from the plant targeted for transformation or from truncated native chromosomes.

### PRIORITY CLAIMS AND REFERENCE TO RELATED APPLICATIONS

This application claims the benefit of priority of U. S. Provisional Patent Application 60/912,032, filed on 16 Apr. 2007, which is incorporated herein by reference in its entirety.

### FIELD OF THE INVENTION

Disclosed herein are transgenic plants and seeds with multiple transgenes on a chromosome for providing traits, methods of making and using such transgenic plants and seeds.

### BACKGROUND OF THE INVENTION

A transgenic tomato was commercially introduced in 1994 followed by transgenic cotton, soybean, corn, potato, canola, papaya, and squash. The transgenic crops that were widely adopted in the first decade had traits that provided environmental, social and economic impact and included herbicide tolerant soybeans, corn and cotton, insect resistant cotton, corn and potato, and virus resistant papaya, squash and potato. These traits permitted significant reductions in pesticide use resulting in substantial environmental benefits in terms of improved soil and water quality, reduced fuel consumption in agriculture and improved soil conservation. With lower costs of production and higher yields farm income also increased. In the first decade it is estimated that herbicide tolerant soybeans globally contributed over $9 billion dollars to increased net farm income while reducing pesticide usage by over 40 million kilograms. All transgenic crops combined globally contributed about $27 billion to increased net farm income while reducing pesticide applications by about 170 million kilograms. With the introduction of insect tolerant cotton India in the period of five years went from a major cotton importer to become one of the world's largest cotton exporters. Improvements in transgenic plants involve stacking traits, e.g. herbicide tolerance and insect resistance and adding resistance to multiple insects. Farmers benefiting from the benefits of transgenic crops are interested in the addition of still other traits, e.g. nematode resistance, enhanced amino acid levels, drought tolerance, increased yield resulting from specific trait imparting transgenes. Such traits will be combined by breeding plants with new transgenic events with plants having existing transgenes. With Mendellian segregation the expectation for a homozygous plant having all desired transgenes combined is (¼)ⁿ, where n is the number of different chromosomal loci with transgenes. If there are 8 loci, the odds of a successful combination of all eight transgenes, value of (¼)ⁿ, is 1/65,536. Moreover the acreage needed for such breeding also increase by an exponential factor.

Stacking additional traits into crops by combining transgenes would be greatly facilitated if the number of loci can be reduced. One approach to providing multiple transgenes in a single locus of a native chromosome is to use site directed transgene insertions using site-specific integrase recombinase systems such as the CRE/lox system from bacteriophage P1 (U.S. Pat. No. 4,959,317; U.S. Pat. No. 5,658,772), the FLP/frt system from yeast (Golic and Lindquist, Cell, 59:499-509, 1989), the Pin recombinase of E. coli (Enomoto, et al., J. Bacteriol., 156(2):663-668, 1983), the Gin/gix recombinase of phage Mu (Maeser et al., Mol. Gen. Genet., 230(1-2):170-176, 1991) and the R/RS system of the pSR1 plasmid from Xygosaccharomyces rouxii (Onouchi et al., Nuc. Acids Res., 19:6373-6378, 1991).

Another approach to providing multiple transgenes in a single locus is to transform a plant cell with a recombinant DNA construct comprising multiple contiguous transgenes. See for instance, US Patent Application Publication 20040045051A1 disclosing transformation of plant cells using plasmids having up to five transgenes.

Alternatively, the multiple transgenes can be provided on a recombinant chromosome that can be inserted into a plant nucleus. Copenhaver et al. disclose in U.S. Pat. No. 7,193,128 constructing recombinant mini chromosomes with transgenes for a variety of crop traits. Such mini chromosomes have centromere DNA that is native to the target crop. Ananiev et al. disclose in U.S. Patent Application Publication 2007/0271629 A1 alternative artificial plant minichromosomes with a functional centromere that specifically binds to centromere protein C.

Yu et al. disclose in U.S. Patent Application Publication 2007/0300331 A1 artificial plant minichromosomes generated in planta by telomere-mediated truncation of native chromosomes. Engineered minichromosomes were constructed by inserting multiple genes, e.g. by site specific recombination, into a truncated native chromosome.

### SUMMARY OF THE INVENTION

This invention provides transgenic plants, fruit and seed having transgenes on one or more recombinant chromosomes. Especially useful aspects of the invention provide transgenic plants and seed having a plurality of transgenes, e.g. at least four transgenes, on a small recombinant chromosome. A transgene comprises recombinant DNA that is transcribed as messenger RNA encoding a protein or RNA for suppressing expression of a gene or both. In one aspect of the invention the recombinant chromosome can be derived from a native chromosome that has had a substantial number of native genes removed, e.g. except for genes regulating the chromosome such as genes for regulating, maintaining or imparting topological or chromatin structure, molecular integrity or stability of gene expression or inheritance. In another aspect of the invention the recombinant chromosome can be synthesized from native chromosome elements, e.g. centromeric DNA and telomeric DNA derived native centromere and telomere from a plant. Recombinant chromosomes can be circular, i.e. having centromeric DNA without telomeric DNA. Recombinant chromosomes can be linear, i.e. having both centromeric and telomeric DNA. Transgenes can be provided at one or more loci, e.g. at a single locus or at two or more loci on the same or opposing sides of centromeric DNA.

Embodiments of transgenic plants and seeds provided by this invention comprise multiple transgenes, e.g. at least four or more, for instance at least five or six transgenes on a recombinant chromosome. The invention is especially advantageous when at least seven, eight, nine or more transgenes are to be provided on a chromosome. In aspects of the invention the transgenes on one chromosome are in a single locus; in other aspects of the invention the transgenes on one chromosome are in two or more loci on a chromosome.

Aspects of the inventions provide transgenic plants, fruit and seed with multiple transgenes on a chromosome where the transgenes provide in the plant, fruit or seed one or more traits selected from the group consisting of herbicide tolerance, insect resistance, nematode resistance, viral resistance, tolerance to water deficit, tolerance to nitrogen deficit, enhanced amino acid level in seed, enhanced starch level in seed, enhanced oil level in seed, modified oil composition, and increased yield as compared to a control plant without the transgene associated with the trait. Such traits are provided by transgenes comprising DNA encoding a protein or DNA that is transcribed as dsRNA for gene suppression as more particularly identified herein. In a preferred embodiment the multiple genes are on a recombinant chromosome

Preferred aspects of the invention provide transgenic corn plants and seed having on at least one heterologous chromosome one or more transgenes, e.g. at least four transgenes selected from the group consisting of a transgene that provides glyphosate herbicide tolerance, a transgene that provides dicamba herbicide tolerance, a transgene that provides glufosinate herbicide tolerance, a transgene that provides lepidopteran insect resistance, a transgene that provides coleopteran insect resistance, a transgene that provides hemipteran or homopteran insect resistance, a transgene that provides nematode resistance, a transgene that provides tolerance to water deficit, a transgene that provides tolerance to nitrogen deficit, a transgene that provides enhanced amino acid level in seed, and a transgene that provides enhanced yield. More preferred are transgenic corn plants and seed having at least five or six or more of such transgenes on one chromosome. Even more preferred are transgenic corn plants and seed having at least seven, eight or nine or more transgenes on one chromosome.

Preferred aspects of the invention provide transgenic soybean plants and seed having on at least one heterologous chromosome one or more transgenes, e.g. at least four transgenes selected from the group consisting of a transgene that provides glyphosate herbicide tolerance, a transgene that provides dicamba herbicide tolerance, a transgene that provides glufosinate herbicide tolerance, a transgene that provides insect resistance, a transgene that provides nematode resistance, a transgene that provides hemipteran or homopteran insect resistance, a transgene that provides tolerance to nitrogen deficit, a transgene that provides tolerance to water deficit, a transgene that provides modified oil composition, and a transgene that provides enhanced yield. More preferred are transgenic soybean plants and seed having at least five or six or more of such transgenes on one chromosome. Even more preferred are transgenic soybean plants and seed having at least seven, eight or nine or more transgenes on one chromosome.

Still other preferred aspects of the invention provide transgenic cotton plants and seed having on at least one heterologous chromosome one or more transgenes, e.g. at least four transgenes, selected from the group consisting of a transgene that provides glyphosate herbicide tolerance, a transgene that provides dicamba herbicide tolerance, a transgene that provides glufosinate herbicide tolerance, a transgene that provides lepidopteran insect resistance, a transgene that provides coleopteran insect resistance, a transgene that provides hemipteran or homopteran insect resistance, a transgene that provides tolerance to nitrogen deficit, a transgene that provides tolerance to water deficit, and a transgene that provides enhanced yield. More preferred are transgenic cotton plants and seed having at least five or six of such transgenes on one chromosome. Even more preferred are transgenic cotton plants and seed having at least seven, eight or nine or more transgenes on one chromosome.

Still yet other preferred aspects of the invention provide transgenic rapeseed plants and seed having on at least one heterologous chromosome one or more transgenes, e.g. at least four transgenes, selected from the group consisting of a transgene that provides tolerance to nitrogen deficit, a transgene that provides glyphosate herbicide tolerance, a transgene that provides dicamba herbicide tolerance, a transgene that provides glufosinate herbicide tolerance, a transgene that provides insect resistance, a transgene that provides hemipteran or homopteran insect resistance, a transgene that provides tolerance to nitrogen deficit, a transgene that provides tolerance to water deficit, and a transgene that provides enhanced yield. More preferred are transgenic rapeseed plants and seed having at least five or six of such transgenes on one chromosome. Even more preferred are transgenic rapeseed plants and seed having at least seven, eight or nine or more transgenes on one chromosome.

Another aspect of the invention provides transgenic vegetable plant and fruit having on at least one heterologous chromosome one or more transgenes, e.g. to provide virus resistance, insect resistance, nematode resistance, herbicide tolerance, water deficit tolerance, and/or nitrogen deficit tolerance.

### DETAILED DESCRIPTION OF PREFERRED EMBODIMENTS

As used herein corn means any variety of Zea mays, soybean means any variety of Glycine max, cotton means any variety of Gossypium hirsutum or Gossypium barbadense, and rapeseed means any variety of Brassica napus including canola.

The production of hybrid corn seed by introgressing multiple traits by breeding is well know by persons of ordinary skill in the art; specific instructions are found in International Application WO 05/033192 A2.

The construction of transgenes using regulatory DNA suitable for specific plants, e.g. monocots and dicots, is well known by persons of ordinary skill in the art; specific instructions is found in US Patent Application Publications 20054/0115642 A1 and 2006/0147961 A1. Methods and materials for recombinant DNA that is expressed as RNA in a plant for suppressing a gene is well known by persons of ordinary skill in the art; specific materials and methods are found in US Patent Application Publication 2007/0011775 A1.

Methods of using recombinase enzymes for site specific integration of transgenes into a plant native chromosome is well known by persons of ordinary skill in the art; specific materials and methods are found in U.S. Pat. No. 6,750,379.

Technology for transforming plant cells by microprojectile bombardment with particles coated with recombinant DNA is well known by persons of ordinary skill in the art; specific instructions are found in U.S. Pat. Nos. 5,015,580 (soybean); 5,550,318 (corn); 5,538,880 (corn); 5,914,451 (soybean); 6,160,208 (corn); 6,399,861 (corn) and 6,153,812 (wheat) and Agrobacterium-mediated transformation is described in U.S. Pat. Nos. 5,159,135 (cotton); 5,824,877 (soybean); 5,591,616 (corn); and 6,384,301 (soybean).

DNA for providing a wide variety of traits in transgenic plants is well known to persons of ordinary skill in the art; more specific DNA for specific traits is found in the following described references. An even more comprehensive list of DNA encoding proteins associated with a plurality of diverse traits is found in International Application [PCT US 2007/080323 filed Oct. 3, 2007].

DNA encoding proteins that impart herbicide tolerance is found in U.S. Pat. No. 4,769,061 (mutant 5-enolpyruvylshikimate-3-phosphate synthase for glyphosate herbicide tolerance), U.S. Pat. No. 5,627,061 (mutant 5-enolpyruvylshikimate-3-phosphate synthase for glyphosate herbicide tolerance), U.S. Pat. No. 5,463,175 (glyphosate oxido-reductase for glyphosate herbicide tolerance), U.S. Pat. No. 5,646,024 (phosphinothricin acetyltransferase for glufosinate herbicide tolerance), U.S. Pat. No. 5,767,366 (a mutant acetolactate synthase for imidazolinone herbicide tolerance), U.S. Pat. No. 4,810,648 (haloarylnitrilase for bromoxynil herbicide tolerance), U.S. Pat. No. 6,414,222 (acetyl-coenzyme A carboxylase for cyclohexanedione or aryloxyphenoxypropanoic acid herbicide tolerance), U.S. Pat. No. 5,597,717 (modified dihydropteroate synthase for sulfonamide herbicide resistance), US Patent Application Publication 2003/0083480A1 (glyphosate-N-acetyl transferase for glyphosate herbicide tolerance), 2003/0115626A1 (dicamba mono-oxygenase for dicamba herbicide tolerance), 2004/0200874A1 (glyphosate decarboxylase for glyphosate herbicide tolerance). See also International Application WO 99/27116 (2,2-dichloropropionic acid dehalogenase for dalapon herbicide resistance).

Bacterial DNA encoding proteins that impart insect resistance, e.g. the Bacillus thuringiensis Cry1A(b), Cry1A(c), Cry3Aa, Cry1Ca, and Cry2Aa delta-endotoxins, for use in transgenes are found in U.S. Pat. Nos. 5,500,365 and 5,689,052. Native sequence of other Bacillus endotoxins is known and can be modified for effective expression in plants. For controlling lepidopteran insects transgenes in plants can comprise DNA encoding a Bacillus thuringiensis Cry1Aa, Cry1Ab, Cry1Ac, Cry1Ba, Cry1Bb, Cry1Ca, Cry2Aa, Cry2Ab, TIC900, or Cry9toxins, VIP proteins including VIP1, VIP2, and particularly VIP3, and particularly including VIP3A, and variants thereof, and insecticidal hybrids including proteins such as Cry1A. 105 and the like. For controlling coleopteran insects transgenes in plants can comprise DNA encoding a Bacillus thuringiensis Cry3A and Cry3B toxins, and variants of these, Cry 3C, Cry 34 in combinations with Cry 35 (i.e., PS149B1), ET33 in combination with ET34, ET29, TIC901, TIC1201, TIC407, TIC435, and TIC417 toxins and variants and combinations thereof, ET29 in combination with TIC810, ET70, combinations of ET80 and ET76, and TIC851 and the like. For controlling hemipteran and homopteran insects transgenes in plants can comprise DNA encoding a combination of ET29 and TIC810 toxins as well as other insecticidal toxins known in the art. The disclosure of DNA encoding ET37, TIC810 and TIC812 proteins from Bacillus thuringiensis, and DNA for use in expressing TIC809, ET37, TIC810 and TIC812, and fusions of various insecticidally effective combinations of these proteins such as TIC 127 in plants is disclosed in International Application WO 07/027776. Also disclosed are methods of making and using the DNA and the proteins in the development of transgenic plant cells and transgenic plants exhibiting improved insect resistance against Coleopteran insects including Western Corn Rootworm (Dibrotica virgifera virgifera), Southern Corn Rootworm (Dibrotica undecempunctata), Northern Corn Rootworm (Diabrotica barberi), Mexican Corn Rootworm (Diabrotica virgifera zeae), Brazilian Corn Rootworm (Diabrotica balteata) and Brazilian Corn Rootworm complex (Diabrotica viridula and Diabrotica speciosa), and against Hemipteran insects such as Lygus bugs. US Patent Publication 2007/022897 discloses DNA encoding endotoxins that are toxic to lepidopteran and coleopteran insects.

Transgenes for controlling nematodes can comprise DNA for expressing in plants RNA that is designed to suppress a gene in the nematode, e.g. VATPase or a major sperm protein, or DNA for expressing in plants a Bacillus thuringiensis Cry5, Cry6 or Cry21 endotoxin.

DNA in plants for controlling virus infections is found in U.S. Pat. No. 6,608,241 (viral coat protein). The production of RNA in a plant cell for suppression of a gene in a virus, e.g. dsRNA targeted to a viral coat protein is an alternative method for providing viral resistance, e.g. resistance to geminiviruses including a tomato yellow leaf curl virus (Genbank reference AF024715, EF54894, AJ132711, NC_{—}004611, NC_{—}004648, AF130415), tomato rugose virus (Genbank reference AY029750), pepper huasteco yellow vein virus (Genbank reference NC_{—}001359), pepper golden mosaic virus (Genbank reference NC_{—}004101), beet severe curly top virus (Genbank reference NC_{—}004754) and resistance to tospoviruses including capsicum chlorosis virus (Genbank reference DQ355974), chrysanthemum stem necrosis virus (Genbank reference AF067068), groundnut bud necrosis virus (Genbank reference AY426316), groundnut ring spot virus (Genbank reference S54327), impatiens necrotis spot virus (Genbank reference DQ523598), peanut yellow spot virus (Genbank reference AY529714), Thailand tomato topsovirus (Genbank reference AF13440), tomato chlorotic spot virus (Genbank reference S54325), tomato spotted wilt virus (Genbank reference DQ523599 and X61799).

Recombinant DNA for imparting water deficit tolerance by expression in plants of a protein with a cold shock domain, a Hap3 transcription factor, a cold binding factor, a 14-3-3 protein, a C terminal processing protease (CtpA), or a combination thereof is found in US Patent application Publications 2003/0233680A1 (cold binding factors), 2005/0097640A1 (cold shock proteins), 2005/0022266A1 (Hap3 transcription factor), International Application WO 04/053055 (14-3-3 proteins) and Oelmuller et al., J. Biol Chem., 1966, Sep 6;271(36):21848-52 (Arabidopsis CtpA).

Recombinant DNA for imparting nitrogen deficit tolerance by expression in plants of a protein with a magnesium transporter protein, a rubisco activase, an alanine aminotransferase as disclosed in US Patent Application Publication 2007/0294782 A1, a chlorate transporter or a translation initiation factor E1F-4F as disclosed in US Patent Application Publication 2005/0108791 A1, or a combination thereof.

Recombinant DNA for imparting enhanced amino acid level in seed by expression in plants of RNA for suppression of an amino acid catabolyte or by expression of an amino acid synthase or by a combination thereof is found in US Patent Application Publication 2005/019344A1.

The recombinant chromosomes of this invention can be synthesized ex planta using plant specific centromeres, e.g. from corn, rapeseed (canola), soybean, and tomato, as disclosed in US Patent Application Publication 2005/0241606 A1 or in US Patent Application Publication 2007/0271629 A1. The use of methylated nucleic acid segments to isolate centromere from a plant for construction of a plant specific recombinant chromosome is found in U.S. Pat. No. 6,649,347. Such recombinant chromosomes can be loaded multiple transgenes by a variety of known methods such as site specific recombination Recombinant chromosomes with multiple transgenes can be introduced into plant cells by

The recombinant chromosomes of this invention can alternatively be produced in planta by telomere-associated chromosomal truncation, e.g. by truncating a corn B chromosome, as disclosed by Yu et al. in US Patent Application Publication 2007/0300331 A1. Multiple transgenes can be inserted into such truncated chromosomes by site specific recombination methods.

The following examples serve to illustrate aspects and embodiments of the invention.

### EXAMPLE 1

This example illustrates the preparation of transgenes with recombinant DNA comprising at the 5′ end a promoter DNA operably linked to DNA for imparting a trait, i.e. DNA for coding RNA for gene suppression or to DNA for expressing a protein, followed at the 3′ end by regulatory DNA, e.g. for polyadenylation. Separate transgenes have unique promoter DNA and polyadenylation DNA.

A transgene is prepared for imparting glyphosate herbicide tolerance with recombinant DNA for expressing a mutant 5-enolpyruvylshikimate-3-phosphate synthase (EPSPS transgene). A transgene is prepared for imparting glyphosate herbicide tolerance with recombinant DNA for expressing a glyphosate-N-acetyl transferase (GAT transgene). A transgene is prepared for imparting dicamba herbicide tolerance with recombinant DNA for expressing a dicamba mono-oxygenase (dicamba transgene). A transgene is prepared for imparting glufosinate herbicide tolerance with recombinant DNA for expressing a phosphinothricin acetyltransferase (pat transgene). A transgene is prepared for imparting lepidopteran insect resistance with recombinant DNA for expressing a Bacillus thuringiensis Cry1A(b) endotoxin (Cry1A(b) transgene). A transgene is prepared for imparting lepidopteran insect resistance with recombinant DNA for expressing a Bacillus thuringiensis Cry2Aa endotoxin (Cry2Aa transgene). A transgene is prepared for imparting coleopteran insect resistance with recombinant DNA for expressing a Bacillus thuringiensis Cry3A endotoxin (Cry3A transgene). A transgene is prepared for imparting hemipteran and homopteran insect resistance with recombinant DNA for expressing a Bacillus thuringiensis ET29 and TIC810 endotoxins (ET29+TIC810 transgene). A transgene is prepared for imparting soybean cyst nematode resistance with recombinant DNA for expressing an RNA that becomes a dsRNA targeting a VATPase gene of soybean cyst nematode (VATPase transgene). A transgene is prepared for imparting water deficit tolerance with recombinant DNA for expressing a cold shock protein from Bacillus subtilis (cspB transgene). A transgene is prepared for imparting water deficit tolerance with recombinant DNA for expressing an Arabidopsis CtpA protein (CtpA transgene). A transgene is prepared for imparting nitrogen deficit tolerance with recombinant DNA for expressing a translation initiation factor E1F-4F (E1F-4F transgene). A transgene is prepared for imparting enhanced lysine amino acid level with recombinant DNA for expressing a dihydropicolinate synthase in the lysine synthase pathway (dhps transgene). A transgene is prepared for imparting enhanced lysine amino acid level with recombinant DNA for expressing an RNA that becomes a dsRNA targeting a lysine ketoglutarate saccharopine dehydrogenase (LKRSDH transgene). A transgene is prepared for imparting modified oil with recombinant DNA for expressing an RNA that becomes a dsRNA targeting a delta 12 desaturase (delta 12 transgene). A transgene is prepared for imparting soybean cyst nematode resistance with recombinant DNA for expressing an RNA that becomes a dsRNA targeting a soybean cyst nematode major sperm protein (SCN msp transgene). A transgene is prepared for imparting tomato rugose virus resistance with recombinant DNA for expressing an RNA that becomes a dsRNA targeting a coat protein targeted to the tomato rugose geminivirus (trg transgene). A transgene is prepared for imparting tomato chlorotic spot virus resistance with recombinant DNA for expressing an RNA that becomes a dsRNA targeting a coat protein targeted to the tomato chorotic spot topsovirus (tcs transgene).

### EXAMPLE 2

This example illustrates the production of recombinant chromosomes for producing transgenic corn plants. A plurality of circular small chromosomes with centromeric DNA derived from a native corn chromosome and a plurality of linear small chromosomes with centromeric and telomeric DNA derived from a native corn chromosome are prepared as disclosed in US Patent Application Publication 2005/0241606 A1.

A plurality of circular small chromosomes are modified by adding transgenes prepared in Example 1 producing
(a) circular corn recombinant chromosome 2-1 with a VATPase transgene and a pat gene,
(b) circular corn recombinant chromosome 2-2 with an EPSPS transgene, a Cry1A(b) transgene, a Cry2Aa transgene and a cspB transgene,
(c) circular corn recombinant chromosome 2-3 with an EPSPS transgene, a Cry1A(b) transgene, a Cry2Aa transgene, a Cry3A transgene, a dhps transgene and a cspB transgene,
(d) circular corn recombinant chromosome 2-4 with an EPSPS transgene, a pat transgene, a Cry1A(b) transgene, a Cry2Aa transgene, a Cry3A transgene, a Hap3 transgene and a cspB transgene, and
(e) circular corn recombinant chromosome 2-5 with a GAT transgene, a Cry1A(b) transgene, a Cry2Aa transgene, a Cry3A transgene, a dhps transgene, an LKRSDH transgene, a CtpA transgene and a cspB transgene.

A plurality of linear small chromosomes are modified by adding transgenes prepared in Example 1 producing
(a) linear corn recombinant chromosome 2-6 with a delta 12 transgene on one side of centromeric DNA and a GAT transgene on the opposing side of the transgene,
(b) linear corn recombinant chromosome 2-7 with an EPSPS transgene, a Cry1A(b) transgene on one side on centromeric DNA and a Cry2Aa transgene and a cspB transgene on the other side of centromeric DNA,
(c) linear corn recombinant chromosome 2-8 with an EPSPS transgene, a Cry1A(b) transgene, a Cry2Aa transgene, a Cry3A transgene on one side of centromeric DNA and a dhps transgene, E1F-4F transgene and a cspB transgene on the other side of centromeric DNA,
(d) linear corn recombinant chromosome 2-9 with an EPSPS transgene, a pat transgene, a Cry1A(b) transgene, a Cry2Aa transgene, a Cry3A transgene, a Hap3 transgene and a cspB transgene all on one side of centromeric DNA,
(e) linear corn recombinant chromosome 2-10 with an EPSPS transgene, a Cry1A(b) transgene, a Cry2Aa transgene, a Cry3A transgene on one side of centromeric DNA and dhps transgene, an LKRSDH transgene, a CtpA transgene and a cspB transgene on the other side of centromeric DNA, and
(f) linear corn recombinant chromosome 2-11 with an GAT transgene, a Cry1A(b) transgene, a Cry2Aa transgene, a Cry3A transgene on one side of centromeric DNA and dhps transgene, an LKRSDH transgene, a CtpA transgene and a cspB transgene on the other side of centromeric DNA,

### EXAMPLE 3

This example illustrates the production of a transgenic corn plants with transgenes on a heterologous chromosome.

Each of the circular corn recombinant chromosomes 2-1 through 2-5 and linear corn recombinant chromosomes 2-6 through 2-11 prepared in Example 2 are separately duplicated and applied to inert microparticles which are bombarded into corn tissue. Using an herbicide tolerance transgene as a selectable marker, transgenic events are grown into plantlings which are screened for single copy of the corn recombinant heterologous chromosome. Transgenic plantlings with a single copy of a corn recombinant chromosome are grown into full plants which are self pollinated to produce transgenic seed with a recombinant chromosome. Progeny transgenic seed is used to produce transgenic plants which are crossed with non-transgenic corn lines to produce hybrid transgenic seed and plants having the traits imparted by the up to nine transgenes. Progeny transgenic seed is also used to produce transgenic plants which are crossed with transgenic corn lines, e.g. having recombinant DNA with a transgene on a native chromosome, to produce hybrid transgenic seed and plants having the traits imparted by the up to ten transgenes.

Each of the circular corn recombinant chromosome 2-1 and the linear corn recombinant chromosomes 2-6 prepared in Example 2 are separately duplicated and applied to inert microparticles which are bombarded into corn tissue. Using the herbicide tolerance transgenes as a selectable marker, transgenic events are grown into plantlings which are screened for single copy of each of the corn recombinant heterologous chromosomes. Transgenic plantlings with a single copy of each corn recombinant chromosome are grown into full plants which are self pollinated to produce transgenic seed with the recombinant heterologous chromosomes. Progeny transgenic seed is used to produce transgenic plants which are crossed with non-transgenic corn lines to produce hybrid transgenic seed and plants having the traits imparted by the two transgenes. Progeny transgenic seed is also used to produce transgenic plants which are crossed with transgenic corn lines, e.g. having recombinant DNA with a transgene on a native chromosome, to produce hybrid transgenic seed and plants having the traits imparted by the transgenes.

### EXAMPLE 4

This example illustrates the production of recombinant chromosomes for producing transgenic soybean plants. A plurality of circular small chromosomes with centromeric DNA derived from a native soybean chromosome and a plurality of linear small chromosomes with centromeric and telomeric DNA derived from a native soybean chromosome are prepared as disclosed in US Patent Application Publication 2005/0241606 A1.

A plurality of circular small chromosomes are modified by adding transgenes prepared in Example 1 producing
(a) circular soybean recombinant chromosome 4-1 with a delta 12 transgene and a GAT transgene,
(b) circular soybean recombinant chromosome 4-2 with an EPSPS transgene, a VATPase transgene, a CtpA transgene, a Hap3 transgene and a cspB transgene,
(c) circular soybean recombinant chromosome 4-3 with an EPSPS transgene, a VATPase transgene, a delta 12 transgene, a CtpA transgene, a Hap3 transgene and a cspB transgene
(d) circular soybean recombinant chromosome 4-4 with an EPSPS transgene, a dicamba transgene, a VATPase transgene, a delta 12 transgene, a CtpA transgene, a Hap3 transgene and a cspB transgene, and
(e) circular soybean recombinant chromosome 4-5 with an EPSPS transgene, a dicamba transgene, a VATPase transgene, a delta 12 transgene, a CtpA transgene, a Happ3 transgene, an E1F-4F transgene and a cspB transgene,.

A plurality of linear small chromosomes are modified by adding transgenes prepared in Example 1 producing
(a) linear soybean recombinant chromosome 4-6 with an SCN msp transgene on one side of centromeric DNA and a pat transgene on the opposing side of the transgene,
(b) linear soybean recombinant chromosome 4-7 with an EPSPS transgene, a pat transgene, an E1F-4F transgene on one side on centromeric DNA and a delta 12 transgene and a Hap3 transgene on the other side of centromeric DNA,
(c) linear soybean recombinant chromosome 4-8 with an EPSPS transgene, a VATPase transgene, an SCN msp transgene on one side of centromeric DNA and a pat transgene, E1F-4F transgene and a cspB transgene on the other side of centromeric DNA,
(d) linear soybean recombinant chromosome 4-9 with an EPSPS transgene, a pat transgene, a VATPase transgene, an SCN msp transgene, a delta 12 transgene, a Hap3 transgene and a cspB transgene all on one side of centromeric DNA,
(e) linear soybean recombinant chromosome 4-10 with an EPSPS transgene, a dicamba transgene, a VATPase transgene, an SCN msp transgene on one side of centromeric DNA and Hap3 transgene, an E1F-4F transgene, a CtpA transgene and a cspB transgene on the other side of centromeric DNA, and
(f) linear soybean recombinant chromosome 4-11 with an EPSPS transgene, a GAT transgene, a VATPase transgene, an SCN msp transgene, a Hap3 transgene, an E1F-4F transgene, a CtpA transgene, a delta 12 transgene and a cspB transgene, all on one side of centromeric DNA.

### EXAMPLE 5

This example illustrates the production of a transgenic soybean plants with recombinant heterologous chromosomes.

Each of the circular soybean recombinant chromosomes 4-1 through 4-5 and linear soybean recombinant chromosomes 4-6 through 4-11 prepared in Example 4 are separately duplicated and applied to inert microparticles which are bombarded into soybean tissue. Using an herbicide tolerance transgene as a selectable marker, transgenic events are grown into plantlings which are screened for single copy of the soybean recombinant chromosome. Transgenic plantlings with a single copy of a soybean recombinant chromosome are grown into full plants which are self pollinated to produce transgenic seed with a recombinant chromosome. Progeny transgenic seed is used to produce transgenic plants which are crossed with non-transgenic soybean lines to produce hybrid transgenic seed and plants having the traits imparted by the transgenes. Progeny transgenic seed is also used to produce transgenic plants which are crossed with transgenic soybean lines, e.g. having recombinant DNA with a transgene on a native chromosome, to produce hybrid transgenic seed and plants having the traits imparted by the transgenes.

Each of the circular soybean recombinant chromosome 4-1 and the linear soybean recombinant chromosomes 4-6 prepared in Example 4 are separately duplicated and applied to inert microparticles which are bombarded into soybean tissue. Using the herbicide tolerance transgene as selectable markers, transgenic events are grown into plantlings which are screened for a single copy of each of the soybean recombinant heterologous chromosomes. Transgenic plantlings with a single copy of each soybean recombinant chromosome are grown into full plants which are self pollinated to produce transgenic seed with the recombinant heterologous chromosomes.

### Example 6

This example illustrates the production of recombinant chromosomes for producing transgenic cotton plants. A plurality of circular small chromosomes with centromeric DNA derived from a native cotton chromosome and a plurality of linear small chromosomes with centromeric and telomeric DNA derived from a native cotton chromosome are prepared as disclosed in US Patent Application Publication 2005/0241606 A1.

A plurality of circular small chromosomes are modified by adding transgenes prepared in Example 1 producing
(a) circular cotton recombinant chromosome 6-1 with an E1F-4F transgene and a GAT transgene,
(b) circular cotton recombinant chromosome 6-2 with an EPSPS transgene, a Cry2Aa transgene, a CtpA transgene, a Hap3 transgene and a cspB transgene,
(c) circular cotton recombinant chromosome 6-3 with an EPSPS transgene, a GAT transgene, a Cry2Aa transgene, a CtpA transgene, a Hap3 transgene and a cspB transgene
(d) circular cotton recombinant chromosome 6-4 with an EPSPS transgene, a dicamba transgene, an E1F-4F transgene, an ET29+TIC 810 transgene, a CtpA transgene, a Hap3 transgene and a cspB transgene, and
(e) circular cotton recombinant chromosome 6-5 with an EPSPS transgene, a dicamba transgene, a Cry1A(b) transgene, a Cry2Aa transgene, a Cry3A transgene, a Hap3 transgene, an EiF-4F transgene and a cspB transgene,.

A plurality of linear small chromosomes are modified by adding transgenes prepared in Example 1 producing
(a) linear cotton recombinant chromosome 6-6 with an EPSPS transgene, a dicamba transgene, a Cry1A(b) transgene and a cspB transgene all on one side of centromeric DNA,
(b) linear cotton recombinant chromosome 6-7 with a GAT transgene, a pat transgene, an E1F-4F transgene on one side on centromeric DNA and a CtpA transgene and a Hap3 transgene on the other side of centromeric DNA,
(c) linear cotton recombinant chromosome 6-8 with an GAT transgene, a dicamba transgene, an E1F-4F transgene on one side of centromeric DNA and a CtpA transgene, a Hap3 transgene and a cspB transgene on the other side of centromeric DNA,
(d) linear cotton recombinant chromosome 6-9 with an EPSPS transgene, a pat transgene, an E1F-4F transgene, an CtpA transgene, a delta 12 transgene, a Hap3 transgene and a cspB transgene all on one side of centromeric DNA,
(e) linear soybean recombinant chromosome 6-10 with an EPSPS transgene, a dicamba transgene, a CtpA transgene, a delta 12 transgene on one side of centromeric DNA and Hap3 transgene, an E1F-4F transgene, a Cry3B transgene and a cspB transgene on the other side of centromeric DNA, and
(f) linear soybean recombinant chromosome 6-11 with an EPSPS transgene, a GAT transgene, a Cry1A(b) transgene, a Cry2Aa transgene, a Cry3A transgene, an E1F-4F transgene, a CtpA transgene, a delta 12 transgene and a cspB transgene, all on one side of centromeric DNA,

### EXAMPLE 7

This example illustrates the production of a transgenic cotton plants with small recombinant chromosomes.

Each of the circular cotton recombinant chromosomes 6-1 through 6-5 and linear cotton recombinant chromosomes 6-6 through 6-11 prepared in Example 6 are separately duplicated and applied to inert microparticles which are bombarded into cotton tissue. Using an herbicide tolerance transgene as a selectable marker, transgenic events are grown into plantlings which are screened for single copy of the cotton recombinant chromosome. Transgenic plantlings with a single copy of a cotton recombinant chromosome are grown into full plants which are self pollinated to produce transgenic seed with a recombinant chromosome. Progeny transgenic seed is used to produce transgenic plants which are crossed with non-transgenic cotton lines to produce hybrid transgenic seed and plants having the traits imparted by the transgenes; the resulting hybrid line is backcrossed with the non-transgenic cotton line to produce an inbred transgenic cotton line. Progeny transgenic seed is also used to produce transgenic plants which are crossed with transgenic cotton lines, e.g. having recombinant DNA with a transgene on a native chromosome, to produce hybrid transgenic seed and plants having the traits imparted by the transgenes.

Each of the circular cotton recombinant chromosome 6-1 and the linear cotton recombinant chromosomes 6-6 prepared in Example 6 are separately duplicated and applied to inert microparticles which are bombarded into cotton tissue. Using the herbicide tolerance transgene as selectable markers, transgenic events are grown into plantlings which are screened for a single copy of each of the cotton recombinant heterologous chromosomes. Transgenic plantlings with a single copy of each cotton recombinant chromosome are grown into full plants which are self pollinated to produce transgenic seed with the recombinant heterologous chromosomes.

### EXAMPLE 8

This example illustrates the production of recombinant chromosomes for producing transgenic rapeseed plants. A plurality of circular small chromosomes with centromeric DNA derived from a native rapeseed chromosome and a plurality of linear small chromosomes with centromeric and telomeric DNA derived from a native rapeseed chromosome are prepared as disclosed in US Patent Application Publication 2005/0241606 A1.

A plurality of circular small chromosomes are modified by adding transgenes prepared in Example 1 producing
(a) circular rapeseed recombinant chromosome 8-1 with a CtpA transgene and n EPSPSB transgene,
(b) circular rapeseed recombinant chromosome 8-2 with an EPSPS transgene, a pat transgene, a CtpA transgene, a Hap3 transgene and a cspB transgene,
(c) circular rapeseed recombinant chromosome 8-3 with an EPSPS transgene, a pat transgene, a delta 12 transgene, a CtpA transgene, a Hap3 transgene and a cspB transgene
(d) circular rapeseed recombinant chromosome 8-4 with an EPSPS transgene, a dicamba transgene, an E1F-4F transgene, a delta 12 transgene, a CtpA transgene, a Hap3 transgene and a cspB transgene, and
(e) circular rapeseed recombinant chromosome 8-5 with an EPSPS transgene, a dicamba transgene, an ET29+TIC810 transgene, a delta 12 transgene, a CtpA transgene, a Hap3 transgene, an E1F-4F transgene and a cspB transgene,.

A plurality of linear small chromosomes are modified by adding transgenes prepared in Example 1 producing
(a) linear rapeseed recombinant chromosome 8-6 with a dicamba transgene and a delta 12 transgene all on one side of centromeric DNA,
(b) linear rapeseed recombinant chromosome 8-7 with an GAT transgene, a pat transgene, an E1F-4F transgene on one side on centromeric DNA and a delta 12 transgene and a Hap3 transgene on the other side of centromeric DNA,
(c) linear rapeseed recombinant chromosome 8-8 with an GAT transgene, an ET29+TIC810 transgene, an delta 12 transgene on one side of centromeric DNA and a pat transgene, E1F-4F transgene and a cspB transgene on the other side of centromeric DNA,
(d) linear rapeseed recombinant chromosome 8-9 with an EPSPS transgene, a pat transgene, a CtpA transgene, an ET29+TIC810 transgene, a delta 12 transgene, a Hap3 transgene and a cspB transgene all on one side of centromeric DNA,
(e) linear rapeseed recombinant chromosome 8-10 with an EPSPS transgene, a dicamba transgene, an ET29+TIC810 transgene, a delta 12 transgene on one side of centromeric DNA and Hap3 transgene, an E1F-4F transgene, a CtpA transgene and a cspB transgene on the other side of centromeric DNA, and
(f) linear rapeseed recombinant chromosome 8-11 with an EPSPS transgene, a GAT transgene, a Cry2Aa [SAME STORY HERE, i.e., do you intend to refer to Cry2Ab?] transgene, an ET29+TIC810 transgene, a Hap3 transgene, an E1F-4F transgene, a CtpA transgene, a delta 12 transgene and a cspB transgene, all on one side of centromeric DNA.

### EXAMPLE 9

This example illustrates the production of a transgenic rapeseed plants with small recombinant chromosomes.

Each of the circular rapeseed recombinant chromosomes 8-1 through 8-5 and linear rapeseed recombinant chromosomes 8-6 through 8-11 prepared in Example 8 are separately duplicated and applied to inert microparticles which are bombarded into rapeseed tissue. Using an herbicide tolerance transgene as a selectable marker, transgenic events are grown into plantlings which are screened for single copy of the rapeseed recombinant chromosome. Transgenic plantlings with a single copy of a rapeseed recombinant chromosome are grown into full plants which are self pollinated to produce transgenic seed with a recombinant chromosome. Progeny transgenic seed is used to produce transgenic plants which are crossed with non-transgenic rapeseed lines to produce hybrid transgenic seed and plants having the traits imparted by the transgenes. Progeny transgenic seed is also used to produce transgenic plants which are crossed with transgenic rapeseed lines, e.g. having recombinant DNA with a transgene on a native chromosome, to produce hybrid transgenic seed and plants having the traits imparted by the transgenes.

Each of the circular rapeseed recombinant chromosome 8-1 and the linear rapeseed recombinant chromosomes 8-6 prepared in Example 8 are separately duplicated and applied to inert microparticles which are bombarded into rapeseed tissue. Using the herbicide tolerance transgene as selectable markers, transgenic events are grown into plantlings which are screened for a single copy of each of the rapeseed recombinant heterologous chromosomes. Transgenic plantlings with a single copy of each rapeseed recombinant chromosome are grown into full plants which are self pollinated to produce transgenic seed with the recombinant heterologous chromosomes.

### EXAMPLE 10

This example illustrates the production of recombinant chromosomes for producing transgenic vegetable plants. A plurality of circular small chromosomes with centromeric DNA derived from a native tomato chromosome and a plurality of linear small chromosomes with centromeric and telomeric DNA derived from a native tomato chromosome are prepared as disclosed in US Patent Application Publication 2005/0241606 A1.

Chromosomes with tomato centromere are modified by adding a transgenes prepared in Example 1 producing
(a) circular tomato recombinant chromosome 10-1 with an tcs transgene and an EPSPSB transgene, and
(b) a linear tomato recombinant chromosome 10-2 with a trv transgene and a pat transgene.

### EXAMPLE 11

This example illustrates the production of a transgenic corn plant with a truncated B chromosome which is prepared in a corn cell as disclosed by Yu et al in US Patent Application Publication 2007/0300331 A1. A variety of transgenes as prepared in Example 1 are introduced into the truncated chromosome by site specific recombination to provide a recombinant chromosome with three separate Bt transgenes, two transgenes for providing drought tolerance and three transgenes for providing herbicide tolerance. The cell is grown on a selectable medium to regenerate a corn seedling which is grown to maturity producing seed from self pollination. The progeny plants containing the heritable recombinant chromosome exhibits resistance to corn root worm and European corn borer, resistance to glyphosate, dicamba and glufosinate herbicides and drought tolerance.

What is claimed is:

1. A transgenic seed or plant of a corn, cotton, rapeseed, soybean or vegetable species having a recombinant chromosome comprising centromere DNA and a plurality of genes, wherein genes on said recombinant chromosome consist essentially of genes for regulating chromosomes and transgenes for imparting traits to said seed or its plant, wherein transgenes comprise recombinant DNA that is transcribed as messenger RNA encoding a protein or RNA for suppressing expression of a gene or both, and wherein said centromere DNA is derived from centromere that is native to said plant; and wherein said plant has none or at least one transgene on a native chromosome.

2. A transgenic seed or plant of claim 1 having on said recombinant chromosome a plurality of at least three, four, five, six, seven eight, or nine transgenes.

3. A transgenic seed or plant of claim 2 wherein said transgenes provide in said plant a trait selected from the group consisting of herbicide tolerance, insect resistance, nematode resistance, viral resistance, tolerance to water deficit, tolerance to nitrogen deficit, enhanced amino acid level in seed, enhanced starch level in seed, enhanced oil level in seed, modified oil composition, and increased yield as compared to a control plant without the transgene associated with said trait.

4. A transgenic seed or plant of claim 3 wherein
(a) said herbicide tolerance is glyphosate tolerance, dicamba tolerance, glufosinate tolerance, pyridine tolerance, or sulfonylurea tolerance,
(b) said insect resistance is resistance to one or more of a lepidopteran insect, a coleopteran insect, a hemipteran insect, and a homopteran insect, or
(c) a combination thereof.

5. A transgenic seed or plant of claim 3 wherein
(a) herbicide tolerance is glyphosate tolerance that is provided by expression of a mutant 5-enolpyruvylshikimate-3-phosphate synthase EPSPS enzyme or a glyphosate oxido-reductase, a glyphosate-N-acetyl transferase, a dicamba mono-oxygenase, a phosphinothricin acetyltransferase, or a mutant acetolactate synthase; reductase, (b) herbicide tolerance is glufosinate tolerance that is provided by expression of a phosphinothricin acetyltransferase; reductase, (c) said herbicide tolerance is dicamba tolerance that is provided by expression of a dicamba mono-oxygenase; reductase, (d) said insect resistance is resistance provided by expression of a Bacillus thuringiensis delta endotoxin protein selected from Cry1Aa, Cry1Ab, Cry1Ac, Cryl Ba, Cry1Bb, Cry1Ca, Cry2Aa, Cry2Ab, Cry3A, Cry3B, Cry3C, Cry9, Cry34 and Cry35 (PS149B1) and variants thereof, a fragments thereof selected from ET33, ET34, ET29, TIC810, TIC900, TIC901, TIC 1201, TIC407, TIC417, PS149B1, a vegetative insecticidal protein selected from VIP1, VIP2, VIP3 and VIP3A and variants thereof, or a hybrid protein Cry1A. 105 and RNA for gene suppression targeting an insect gene;
(e) said nematode resistance is provided by expression of RNA for suppression of a nematode gene, a Bacillus thuringiensis delta endotoxin protein selected from Cry5, Cry 6, or Cry21;
(f) said viral resistance is resistance to a tospovirus or a geminivirus by expression of RNA that forms dsRNA targeted to RNA encoding a coat protein;
(g) said tolerance to water deficit is provided by expression of a protein with a cold shock domain, an NFY transcription factor, a cold binding factor, a 14-3-3 protein, or CtpA;
(h) said tolerance to nitrogen deficit is provided by expression of a magnesium transporter protein, a rubisco activase, an alanine aminotransferase, a chlorate transporter, or a translation initiation factor E1F-4F;
(i) said enhanced amino acid level in seed is provided by expression of RNA for suppression of an amino acid catabolyte or by expression of an amino acid synthase;
(j) said enhanced starch level in seed is provided by expression of RNA for suppression of an amylase or by expression of a starch synthase;
(k) said enhanced oil level in seed is provided by expression of RNA for suppression of a lipase or by expression of an acyltransferase;
(l) said modified oil composition is provided by expression of RNA for suppression of a desaturase or a thioesterase or by expression of a desaturase or a thioesterase;
(m) said increased yield is provided by expression of a transcription factor; or
(n) combinations among and within elements (a) through (k).

6. A transgenic seed or plant of claim 3 wherein said transgenes on said recombinant chromosome are on a single locus on a circular or linear chromosome or are on two loci on opposing sides of centromere DNA on a linear chromosome.

7. A transgenic corn seed or plant of claim 6 having at least four transgenes that are selected from the group consisting of a transgene that provides glyphosate herbicide tolerance, a transgene that provides lepidopteran insect resistance, a transgene that provides coleopteran insect resistance, a transgene that provides hemipteran or homopteran insect resistance, a transgene that provides nematode resistance, a transgene that provides tolerance to water deficit, a transgene that provides enhanced amino acid level in seed, a transgene that provides nitrogen deficit tolerance and a transgene that provides enhanced yield.

8. A transgenic soybean seed or plant of claim 6 having at least four transgenes that are selected from the group consisting of a transgene that provides glyphosate herbicide tolerance, a transgene that provides nematode resistance, a transgene that provides tolerance to water deficit, a transgene that provides enhanced amino acid level in seed, a transgene that provides modified oil composition, a transgene that provides nitrogen deficit tolerance and a transgene that provides enhanced yield.

9. A transgenic cotton seed or plant of claim 6 having at least four transgenes that are selected from the group consisting of a transgene that provides glyphosate herbicide tolerance, a transgene that provides lepidopteran insect resistance, a transgene that provides coleopteran insect resistance, a transgene that provides hemipteran or homopteran insect resistance, a transgene that provides nematode resistance, a transgene that provides tolerance to water deficit, a transgene that provides nitrogen deficit tolerance and a transgene that provides enhanced yield.

10. A transgenic rapeseed seed or plant of claim 6 having at least four transgenes that are selected from the group consisting of a transgene that provides glyphosate herbicide tolerance, a transgene that provides lepidopteran insect resistance, a transgene that provides coleopteran insect resistance, a transgene that provides hemipteran or homopteran insect resistance, a transgene that provides nematode resistance, a transgene that provides tolerance to water deficit, a transgene that provides nitrogen deficit tolerance and a transgene that provides enhanced yield.

11. A transgenic seed or plant of a corn, cotton, rapeseed, soybean or tomato species having one or more recombinant chromosomes, wherein a recombinant chromosome comprises centromere DNA that is derived from centromere that is native to said plant and at least one transgenes for imparting a trait to said seed or its plant, wherein a transgene comprises recombinant DNA that is transcribed as messenger RNA encoding a protein or RNA for suppressing expression of a gene or both, and wherein said transgene imparts a trait that is selected from the group of traits for
(a) glyphosate tolerance that is provided by expression of a mutant 5-enolpyruvylshikimate-3-phosphate synthase EPSPS enzyme or a glyphosate oxido-reductase, a glyphosate-N-acetyl transferase, a dicamba mono-oxygenase, a phosphinothricin acetyltransferase, or a mutant acetolactate synthase;
(b) glufosinate tolerance that is provided by expression of a phosphinothricin acetyltransferase;
(c) dicamba tolerance that is provided by expression of a dicamba mono-oxygenase;
(d) insect resistance that is provided by expression of a Bacillus thuringiensis delta endotoxin protein selected from Cry1Aa, Cry1Ab, Cry1Ac, Cryl Ba, Cry1Bb, Cry1Ca, Cry2Aa, Cry2Ab, Cry3A, Cry3B, Cry3C, Cry9, Cry34 and Cry35 (PS149B1) and variants thereof, a fragments thereof selected from ET33, ET34, ET29, TIC810, TIC900, TIC901, TIC 1201, TIC407, TIC417, PS149B1, a vegetative insecticidal protein selected from VIP1, VIP2, VIP3 and VIP3A and variants thereof, or a hybrid protein Cry1A. 105 and RNA for gene suppression targeting an insect gene;
(e) nematode resistance that is provided by expression of RNA for suppression of a nematode gene, a Bacillus thuringiensis delta endotoxin protein selected from Cry5, Cry 6, or Cry21;
(f) viral resistance that is resistance to a tospovirus or a geminivirus by expression of RNA that forms dsRNA targeted to RNA encoding a coat protein;
(g) tolerance to water deficit that is provided by expression of a protein with a cold shock domain, an NFY transcription factor, a cold binding factor, a 14-3-3 protein, or CtpA;
(h) tolerance to nitrogen deficit that is provided by expression of a magnesium transporter protein, a rubisco activase, an alanine aminotransferase, a chlorate transporter, or a translation initiation factor E1F-4F;
(i) enhanced amino acid level in seed that is provided by expression of RNA for suppression of an amino acid catabolyte or by expression of an amino acid synthase;
(j) enhanced starch level in seed that is provided by expression of RNA for suppression of an amylase or by expression of a starch synthase;
(k) enhanced oil level in seed that is provided by expression of RNA for suppression of a lipase or by expression of an acyltransferase;
(l) modified oil composition that is provided by expression of RNA for suppression of a desaturase or a thioesterase or by expression of a desaturase or a thioesterase; or
(m) increased yield that is provided by expression of a transcription factor.
